(19) United States
(12) Patent Application Publication
(10) Pub. No.: US 2021/0145312 A1
(43) Pub. Date: May 20, 2021

(54) NON-INVASIVE DEVICE AND METHOD FOR SENSING RESPIRATORY PARAMETERS

(71) Applicant: NANOVATION G.S. LTD., Ramat Yishai (IL)

(72) Inventors: Gregory SHUSTER, Haifa (IL); Sagi GLIKSMAN, Ramat Yiahai (IL); Nadav BACHAR, Ramat Yiahai (IL)

(73) Assignee: NANOVATION G.S. LTD., Ramat Yishai (IL)

(21) Appl. No.: 16/616,512

(22) PCT Filed: May 10, 2018
(86) PCT No.: PCT/IL2018/050512

§ 371 Date: Nov. 25, 2019

Related U.S. Application Data
(63) Continuation of application No. 15/604,866, filed on May 25, 2017, now abandoned.

(51) Int. Cl. A61B 5/097; A61B 5/00

(52) U.S. Cl. CPC A61B 5/097; A61B 5/682; A61B 5/6819

(57) ABSTRACT

A non-invasive device for holding one or more respiratory sensors includes a housing anatomically shaped to be attached to a subject's face in proximity to the respiratory orifices, and including one or more flow directing elements for directing at least a portion of the respiratory flow to one or more locations in the housing configured to hold at least one sensor.

### BACKGROUND OF THE INVENTION

Measurement of respiratory flow parameters is one of the tools for assessing the respiratory ability of a patient. The parameters may include: breath temperature, flow rate, volume, pressure, the amount of exhaled non-organic compounds (e.g., water/humidity, CO₂, O₂, etc.) and exhaled volatile organic compounds (VOCs), as well as respiratory function parameters that can be extracted from such measurements, for example respiratory rate, respiratory length and depth, apneas length, time of inhale and exhale and the like. Such measurements are done using either non-direct measurements or direct respiratory sensors that detect/measure/monitor the actual respiratory flow. In order for such direct sensors to operate effectively, the respiratory flow or at least portion of it (inhaled and/or exhaled) is required to pass in the vicinity of the sensors, to enable an interaction between the respiratory flow and the sensing element.

There are several known methods and devices for measuring respiratory flow parameters. One of the known methods requires the subject to intentionally breathe towards a sensor, into or from a mouth-peace, such as in for example, lung function test and spirometry for measuring ventilation and the movement of air into or out of the lungs. For non-ventilated patients these methods can only be applied as non-continuous monitoring in which the patient must be awake, aware and cooperative.

Another known method involves devices that require mounting to the subject's face such that the sensors included in the devices are located in the respiratory flow. One example for such a device is direct capnography. This device is cumbersome and requires placing the sensors in a mask covering the subject's nostrils/mouth. Another example, includes wearable sensors mounted to the subject for example, on face/head, in such way that the sensors are placed around nostrils and near the mouth. Such wearable sensors can be, but are not limited to, temperature, humidity, chemical, pressure and flow sensors. One of the main drawbacks of such wearable sensors is the requirement for precise placement and fine tuning of the sensors' position on the subject's face, in order to align the sensors with the respiratory flow. The alternative is to utilize cannula-like designs that place the sensors inside the subject's nostrils and directly in front of his mouth. This leads to a different drawback: the inconvenience to the subject due to the placement of foreign object in his/her nostrils and on his or her mouth.

Another known method involves devices for capturing and transferring at least a portion of the respiratory flow from the respiratory orifices and transferring, via tubes, the portion of the respiratory flow to external sensors. The main drawback of these sensing devices is that they are not fully wearable and easily portable, because although the capturing and transferring device is wearable, the external sensor is usually large and heavy.

Accordingly, there is a need for a device for measuring respiratory flow that will allow simple, non-obtrusive and robust mounting of one or more respiratory sensors in proximity to a subject's respiratory orifices, to achieve enhanced detection performance, measurement and monitoring of breath, respiration, their mechanical parameters, physical properties, the chemical content of the exhaled/inhaled flow and more.

### SUMMARY OF THE INVENTION

Some embodiments of the invention may be directed to a non-invasive device for holding one or more respiratory sensors. The non-invasive device may include a housing anatomically shaped to be attached to a face of a subject in proximity to the respiratory orifices. In some embodiments, the housing may include: one or more flow directing elements for directing at least a portion of the respiratory flow to one or more predetermined locations, such that, at least one of the predetermined locations may be configured to hold at least one sensor. “Non-invasive device” may mean that the device is not inserted into a respiratory orifice, does not otherwise obstruct a respiratory orifice, and/or is not sealed around a respiratory orifice

In some embodiments, each of the one or more flow directing elements may be or may include at least one open conduit. In some embodiments, the one of the one or more open conduits for guiding the at least a portion of the respiratory flow may be detachably connectable to the housing and may be adapted to guide or direct respiratory flow from and to the mouth over the respiratory sensor. In some embodiments, the one or more open conduits for guiding at least a portion of respiratory flow may be connected to the housing, in a manner that allows a first open conduit to be moved with respect to a second open conduit.

In some embodiments, the one or more flow directing elements may include a recess. In some embodiment's, each recess may be located at the predetermined location and the recess walls may encapsulate the at least one sensor from at least two sides, to prevent or limit physical access to the sensor. In some embodiments, the one or more flow directing elements may be or may include at least one of: a flat surface, a curved surface, a pipe and a combination thereof.

In some embodiments, the one or more flow directing elements may be designed to form at least one of: a controlled environment and a controlled flow regime in proximity to the sensor. In some embodiments, the housing may further include one or more connectors for connecting at least one of: heating and cooling elements. In some embodiments, the housing may further include one or more cavities for holding at least one of: heating and cooling element. In some embodiments, the housing may further include one or more channels for receiving at least one of: a heating element and a cooling element.

In some embodiments, the non-invasive device may further include a mounting mechanism for mounting the non-invasive device to the face of the subject. In some embodiments, the non-invasive device may further include a channel to receive tubing for providing fluids. In some embodiments, the non-invasive device may further include connecting elements for receiving one or more add-on devices.

Some aspects of the invention may be related to a non-invasive device for measuring one or more respiratory flow parameters, comprising: a first sensor for measuring one or more respiratory flow parameters and a housing anatomically shaped to be attached to a face of a subject in proximity to the respiratory orifices. In some embodiments, the housing may include one or more flow directing elements for directing at least a portion of the respiratory flow to at least a first predetermined location, wherein the first predetermined location is configured to hold at least the first sensor.

In some embodiments, the non-invasive device may further include a second sensor and wherein the one or more flow directing elements are for directing at least a portion of the respiratory flow to at least a second predetermined location, wherein the second predetermined location is configured to hold the second sensor. In some embodiments, the first sensor may be for measuring a first type of respiratory flow parameters and the second sensor may be for measuring a second type of respiratory flow parameters. In some embodiments, the first sensor may be for measuring a first type of respiratory flow parameters at the first predetermined location and the second sensor may be for measuring the first type of respiratory flow parameters at the second predetermined location.

Some embodiment of the invention may be related to a method of measuring one or more respiratory flow parameters. The method may include mounting a non-invasive device to a face of a subject. In some embodiments the non-invasive device may include at least one sensor for measuring one or more respiratory flow parameters; and a housing anatomically shaped to be attached to a face of a subject in proximity to the respiratory orifices. In some embodiments, the housing may include one or more flow directing elements for directing at least a portion of the respiratory flow to at least a first predetermined location, wherein the first predetermined location is configured to hold the at least one sensor. In some embodiments, the method may further include receiving measurements from the at least one sensor.

In some embodiments, the method may further include controlling at least one of heating and cooling element included in the non-invasive device, to control the environment in a vicinity to the at least one sensor.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIGS. 1A and 1B are illustrations of back and front views of a non-invasive device for holding one or more sensors according to some embodiments of the invention;

FIG. 2 is an illustration of a non-invasive device for holding one or more sensors according to some embodiments of the invention;

FIG. 3 is an illustration of the non-invasive device for holding one or more sensors of FIG. 1 mounted to a face of a subject according to some embodiments of the invention;

FIG. 4 is a flowchart of a method of measuring one or more respiratory flow parameters according to some embodiments of the invention; and

FIGS. 5A and 5B are graphs presenting humidity and temperature measurements of the respiratory flow measured using a device according to some embodiments of the invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

Some aspects of the invention may be directed to a device for holding one or more respiratory sensors in proximity to a subject's respiratory orifices in order to allow accurate measurements of different parameters of the respiratory flow. The parameters may include both chemical and physical properties of the flow. For example, the parameters may include, flow rate, temperature, volume, pressure, concentration of non-organic compounds (e.g., water/humidity, CO₂, O₂, etc.) and volatile organic compounds in the respiratory flow, and the like. Each of the parameters may be sensed using a designated sensor. One or more designated sensors may be placed in predetermined locations inside the device. The device may include one or more flow directing elements for directing the respiratory flow from the respiratory orifices towards the sensors.

A device according to embodiments of the invention may allow simple mounting of the device to the face of any subject that does not require a unique placement of the device for each specific subject, while allowing highly accurate measurements to be taken by the sensors. The accuracy of the measurements may be due to the controlled environment and flow regimes formed by the directing elements in vicinity of the sensors.

In some embodiments, the non-invasive device may allow directing of a relatively constant portion of the respiratory flow, in an amount sufficient for receiving measurements (readings) from a sensor, through the sensors, without the need to put a mask or other sealing around a subject's nostrils and mouth. The device may provide a controlled environment for the sensors that may allow receiving pf robust and repeatable measurements, as discussed below. In some embodiments, measurements taken using the non-invasive device may also enable (at least partially) quantification of respiratory flow (inhale/exhale flow changes between different breaths), which is not possible with many current solutions. In comparison, only the masks (or other “sealed” methods) are able to provide such repeatable and robust conditions for the measurement and sample delivery.

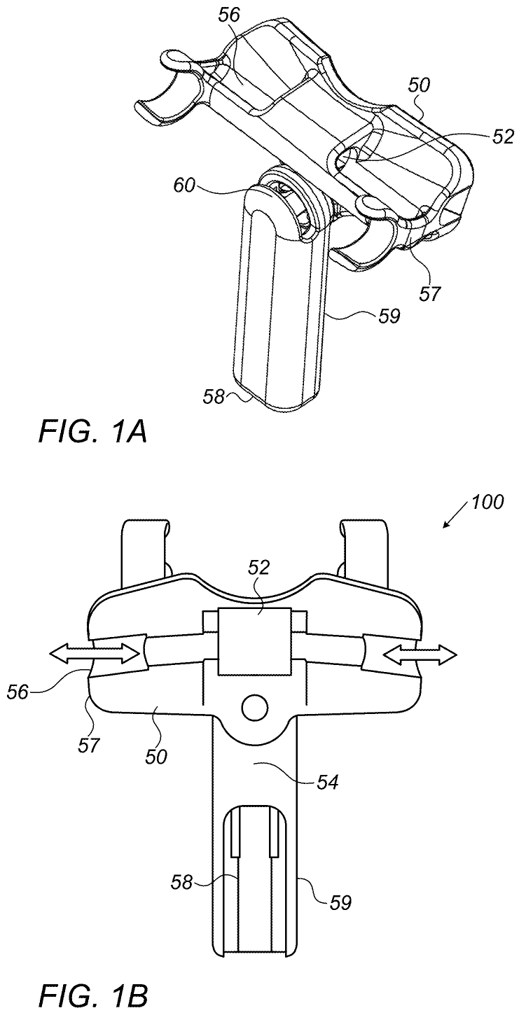

Reference is now made to FIGS. 1A and 1B which are illustrations of front and back views of a non-invasive device 100 for holding one or more sensors according to some embodiments of the invention. Device 100 may include a housing 50 anatomically shaped to be attached or otherwise mounted to a face of a subject in proximity to the respiratory orifices. Housing 50 may include one or more flow directing elements 56 and/or 58 for directing at least a portion of the respiratory flow to one or more predetermined locations in the housing, for example, within flow directing elements 56 and/or 58 or at locations 52 and/or 54 in another example. One or more predetermined locations may be in any place in flow directing elements 56 and/or 58, for example, at the opening of flow directing elements 56 and/or 58, in any location on the walls of flow directing elements 56 and/or 58 and the like. In some embodiments, each of the predetermined locations, for example, locations 52 and/or 54, may be configured to hold at least one sensor. In some embodiments, the predetermined locations may be determined by analyzing the flow pattern from the respiratory orifices through flow directing elements 56 and/or 58 to find the optimal/desired location for placing the sensors. In some embodiments, device 100 may further include the at least one sensor (not illustrated) and any form of communication (not illustrated) for sending measurements taken by the at least one sensor to a controller.

Housing 50 may include or may be made from any suitable material, such as various polymers, ceramics or metals or a combination of more than one material. Housing 50 may be shaped to cause minimal inconvenience to the subject, for example, by being placed external to the respiratory orifices and allowing the subject to eat, drink or the like while wearing device 100. Accordingly, housing 50 may be at most 10 cm wide by 15 cm length, for example, 3 cm by 5 cm, 3.5 cm by 4 cm, 6 cm by 6 cm and 7 cm by 7 cm.

In some embodiments, flow directing elements 56 and 58 may be designed to form at least a controlled environment and/or a controlled flow regime in proximity to the predetermined locations, for example, locations 52 and/or 54. As used herein, the term “controlled environment” may refer to, for example, one or more of: repeatable flow patterns, substantially steady flow rates, substantially steady temperature levels, substantially steady humidity levels and the like. A controlled environment may permit receiving steady measurements from the sensors placed in predetermined locations 52 and 54.

In some embodiments, each flow directing element 56 and/or 58 may be or may include at least one open conduit (as illustrated) or may be or may include a flat surface, a curved surface, a pipe and the like or a combination thereof. In some embodiments, flow directing element 56 may be designed to guide at least a portion of the respiratory flow (e.g., from/to the nose) over a sensor placed in a first predetermined location, for example, location 52. In some embodiments, flow directing element 58 may be designed to guide at least a portion of the respiratory flow (e.g., from/to the mouth) over a sensor placed in a second predetermined location, for example, location 54.

In some embodiments, flow directing elements 58, for example, in the form of an open conduit or any other shape may be detachably connected to housing 50. In some embodiments, flow directing elements 58, for example, in the form of an open conduit may be for guiding respiratory flow from and to the mouth over the respiratory sensor (as illustrated in FIGS. 1A and 1B). For example, a portion 59 of housing 50 that includes flow directing elements 58 and predetermined location 54 may be detachably connectable to a portion 57 of housing 50. Accordingly, upon disconnecting flow directing elements 58 from housing 50, device 100 may include flow directing elements 56 and predetermined location 52, for example, when only measurements of the respiratory flow of the nose are required. Upon reconnecting portion 59 to device 100, measurements from the respiratory flow of the mouth may also be taken by a sensor placed in predetermined location 54. Portion 59 may be detachably connectable to portion 57 using any known way, for example, via a connecting element 60. Connecting element 60 may be, or may include at least one of: a hinge, a rail, a magnet and the like.

In some embodiments, one or more flow directing elements 58, for example, in the form of open conduits or any other shape, may be connected to housing 50, in a manner that allows flow directing element 58 to be moved with respect to flow directing element 56. For example, connecting element 60 may allow portion 59 to move (e.g., shift) or pivot around connecting element 60, from one side of the mouth to the other, for example, to allow the subject to eat or receive medications orally.

In some embodiments, each of one of the predetermined locations, for example, locations 52 and 54, may be configured to hold at least two sensors. The two sensors may be either identical or different. In some embodiments, three, four or more sensors may be located at the predetermined locations in flow directing elements 56 and/or 58. In some embodiments, the predetermined locations, for example, locations 52 and 54 or other locations may be determined by analyzing the flow pattern from the respiratory orifices through flow directing elements 56 and/or 58 to find the optimal location for placing the one or more sensors. The optimal location may be determined as the location at which at least a controlled environment and/or a controlled flow regime may occur during a continuous respiratory parameters measurement. The optimal/desired location may be determined also based on the type of the sensor to be located in the predetermined location and the required measurements to be taken by the sensor. As may be understood by one skilled in the art, the predetermined locations may be at any location in flow directing elements 56 and/or 58 determined based on an analysis of the flow pattern through the flow directing elements and the invention as a whole is not limited to predetermined locations 52 and 54, which are given as an example only.

In some embodiments, one or more flow directing elements 56 and/or 58 may include recesses, for example located at predetermined locations 52 and 54, as illustrated in FIG. 1A. The recesses at locations 52 and 54 may be shaped to support at least one sensor. In some embodiments, the recess at location 52 may be shaped to support a first type of sensor and recess at location 54 may be shaped to support a second type of sensor. In some embodiments, both recesses may be shaped to support the same type of sensor. In some embodiments, the recesses walls at locations 52 and 54 may encapsulate the at least one sensor from at least two sides, to prevent physical access to the sensor. For example, housing 50 may be shaped such that sensors placed in predetermined locations 52 and/or 54 may be covered (as illustrated) while still allowing flow of air to reach the sensors. The cover may protect the sensor from unintentional movement or touch by the subject or a caregiver, protect the sensor from the subject's fluids, and the like.

In some embodiments, housing 50 may further be configured to receive or hold at least one of: heating and cooling elements (not illustrated). The at least one of: heating and cooling elements may be added to device 100 for maintaining a controlled environment in the vicinity of the sensors. The controlled environment may include a controlled temperature, a controlled condensation, controlled humidity and the like. In some embodiments, housing 50 may include connectors (not illustrated) for connecting at least one of: heating and cooling elements to housing 50. In some embodiments, housing 50 may include one or more cavities for holding heating and/or cooling element(s). The one or more cavities may be located within one or more flow directing elements 56 and/or 58 in proximity to the one or more predetermined locations 52 and/or 54. In some embodiments, housing 50 may include one or more channels, for allowing a heating element such as a wire, and/or a cooling element, such as a pipe or Peltier device, to be threaded into housing 50.

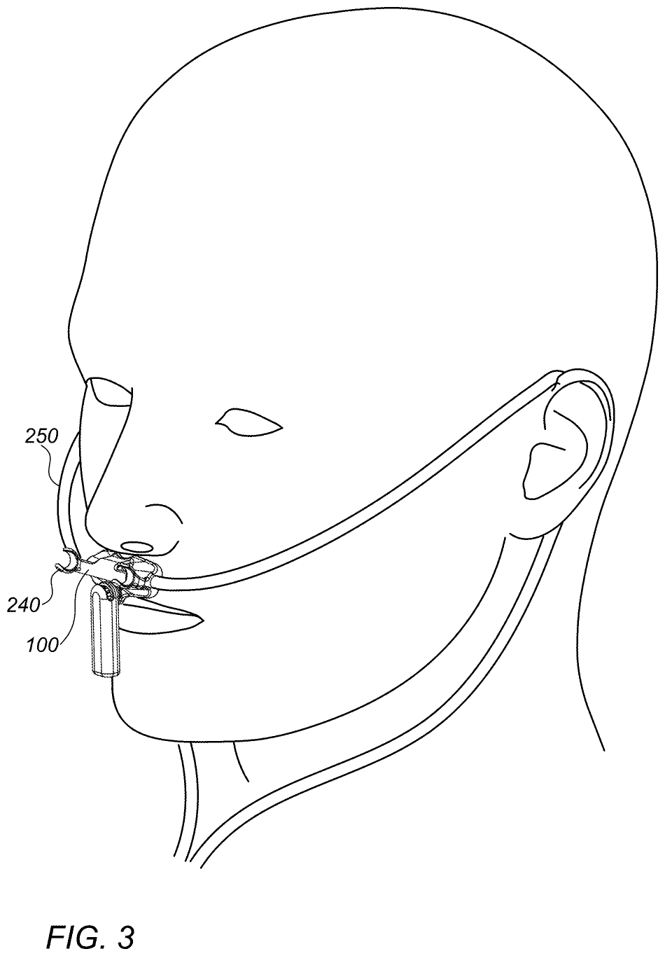

In some embodiments, device 100 may include a mounting mechanism (not illustrated) for mounting device 100 to the subject's face. An example, for a mounting mechanism is given in FIG. 3. In some embodiments, the mounting mechanism may include at least one of, but not limited to: magnets, glue, cords, strings, tubing, glasses-like frame mounting, clips, piercing, elastic bands and the like.

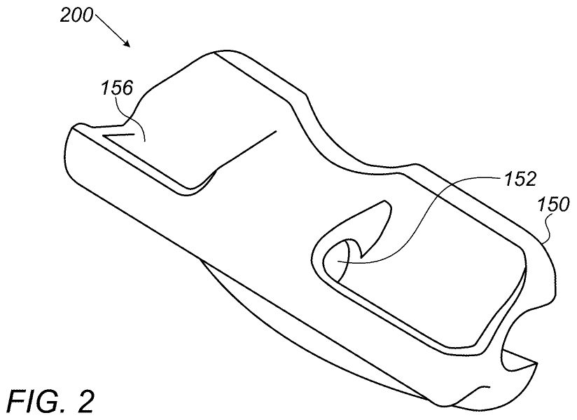

Reference is now made to FIG. 2 which is an illustration of a non-invasive device for holding one or more sensors according to some embodiments of the invention. Device 200 may include a housing 150. Housing 150 may include a single flow directing element 156 for guiding the respiratory flow from and to each nostril over a sensor located at predetermined location 152. Flow directing element 156 may include two open conduits and at least one predetermined location 152, for example, in the form of a recess. Device 200 may further include at least one sensor and a mounting element for mounting device 200 to the face of the subject. As shown, device 200 may include a housing anatomically shaped for attachment to a subject's face.

Reference is now made to FIG. 3, which is an illustration of the non-invasive device for holding one or more sensors of FIG. 1 mounted to a face of a subject according to some embodiments of the invention. Device 100 may be mounted to the face of the subject in proximity to the respiratory orifices using a mounting element, such as for example, wires 250. In some embodiments, wires 250 may be also be the electrical wires for transmitting the signals measured by the one or more sensors located in housing 50 to a controller. In some embodiments, the mounting element may include at least one of: stickers, glue, cords, strings, and elastic bands. In some embodiments, device 100 may include one or more additional connecting elements 240 for receiving one or more add-on devices. The add-on device may include one of: CO₂ sensor, CO₂/O₂/other cannulas, mouth opener, temperature sensor, invasive endoscope, O₂ saturation sensor, a pulse rate sensor and the like. In some embodiments, the add-on devices may be or may include a channel for receiving tubing for providing fluids. The channel may be at least one of: feeding tubes, O₂/air channels and the like.

In some embodiments, non-invasive devices 100 or 200 may further include a first sensor located at a first predetermined location, for example, location 52 or 152 or another first location for measuring one or more respiratory flow parameters. In some embodiments, devices 100 or 200 may further include a second sensor located either at the first predetermined location, for example, location 52 or 152 or at a second predetermined location, for example, location 54 or another second location. In some embodiments, the first sensor may be for measuring a first type of respiratory flow parameter (e.g., humidity) and the second sensor may be for measuring a second type of respiratory flow parameter (e.g., temperature). In some embodiments, the first sensor may be for measuring a first type of respiratory flow parameter (e.g., humidity) at the first predetermined location (e.g., location 52) and the second sensor is for measuring the first type of respiratory flow parameter at the second predetermined location (e.g., location 54). In some embodiments, the first sensor and/or the second sensor may be for measuring: breath's temperature, flow rate, volume, pressure, the amount of non-organic compounds in the breath (e.g., water/humidity, CO₂, O₂, etc.), the amount of volatile organic compounds exhaled from the breath and the like.

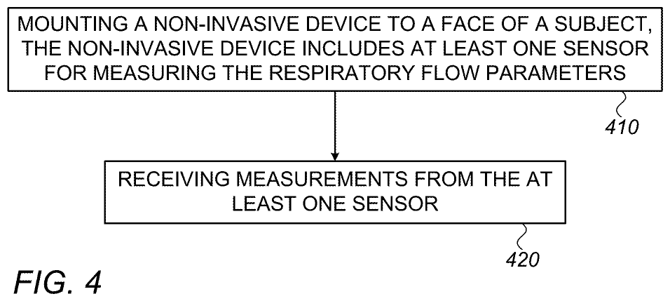

Reference is now made to FIG. 4 which is a flowchart of a method of measuring one or more respiratory flow parameters according to some embodiments of the invention. A non-invasive device, such as device 100 or 200 may be mounted to a face of a subject (as illustrated in FIG. 3), in box 410. Device 100 or 200 may include a housing 50 or 150 anatomically shaped to be attached to a face of any subject in proximity to the respiratory orifices and at least one sensor for measuring the respiratory flow parameters. Each of the sensors may be held in a predetermined location (e.g., locations 52, 54 or 152 or 56 or 58) at one or more flow directing elements (e.g., flow directing elements 56, 58 or 156) for directing at least a portion of the respiratory flow to at least a first predetermined location.

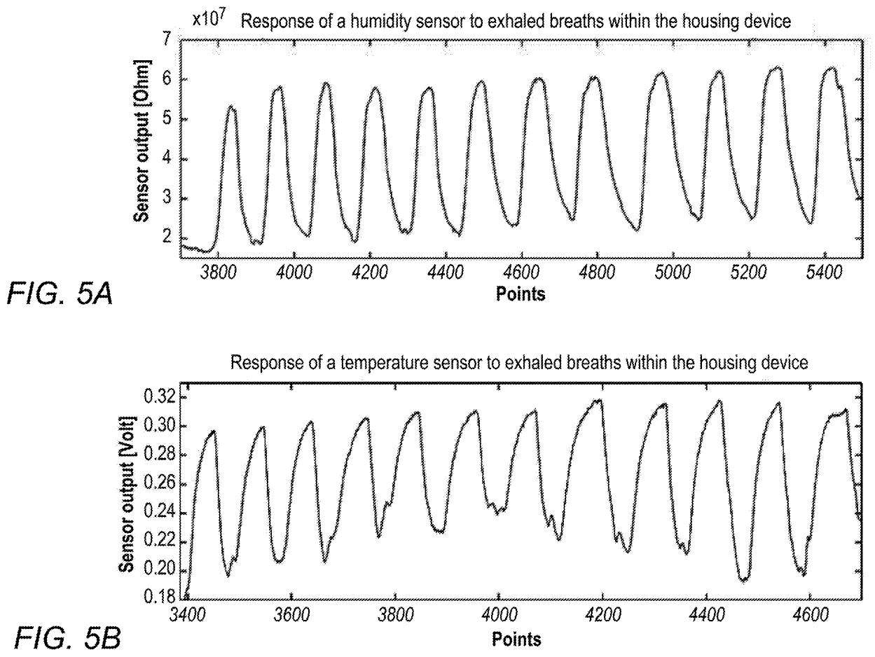

In some embodiments, measurements of respiratory parameters may be received from the at least one sensor, in box 420. The respiratory parameters may include: breath temperature, flow rate, volume, pressure, the amount of non-organic compounds in the breath (e.g., water/humidity, CO₂, O₂, etc.), the amount of volatile organic compounds exhaled from the breath and the like. A controller being in communication with the at least one sensor may receive the measurements and may further process and display the measurements on a display. Examples, for displayed measurements are given in FIGS. 5A and 5B.

Reference is now made to FIGS. 5A and 5B, which are graphs of measurements of response to humidity sensor in Ohms (FIG. 5A) and response to temperature sensor in Volts (FIG. 5B) of the respiratory flow of a subject measured by temperature and humidity sensors held in a device, such as device 100. The graphs show steady repeatable measurements during both the inhale and exhale stages.

Some embodiments of the method may further include heating and/or cooling the environment in the vicinity to the at least one sensor for maintaining a controlled environment. The controlled environment may include a controlled temperature, a controlled condensation, a controlled humidity and the like. In some embodiments cooling and/or heating elements attached, held, threaded to the like, into housing 50 may be activated (e.g., by the controller) to form the controlled environment.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A non-invasive device for holding one or more sensors, comprising:
a housing anatomically shaped to be attached to a face of a subject externally to the respiratory orifices,
wherein the housing comprises:
one or more flow directing elements for directing at least a portion of the respiratory flow to one or more predetermined locations, wherein at least one of the predetermined locations is configured to hold at least one sensor and wherein each of the one or more flow directing elements includes at least one open conduit.

2. (canceled)

3. The non-invasive device according to claim 1, wherein at least one of the one or more flow directing elements include a recess.

4. The non-invasive device according to claim 1, wherein the one or more flow directing elements include at least one of: a flat surface, a curved surface, a pipe and a combination thereof.

5. The non-invasive device of claim 1, wherein the at least one open conduit directing at least a portion of the respiratory flow is detachably connectable to the housing and is configured to guide respiratory flow from and to the mouth over the at least one sensor.

6. The non-invasive device of claim 1, wherein the at least one open conduit for directing at least a portion of the respiratory flow is connected to the housing in a manner that allows a first open conduit to be moved with respect to a second open conduit.

7. The non-invasive device of claim 3, wherein each recess is located at the predetermined location and the recess walls encapsulate the at least one sensor from at least two sides, to prevent physical access to the sensor.

8. The non-invasive device according to claim 1, wherein the one or more flow directing elements are designed to form at least one of: a controlled environment and a controlled flow regime in proximity to the sensor.

9. The non-invasive device according to claim 1, wherein the housing further comprises one or more connectors for connecting at least one of: a heating element and a cooling element.

10. The non-invasive device according to claim 1, wherein the housing further comprises one or more cavities for holding at least one of: a heating element and a cooling element.

11. The non-invasive device according to claim 1, wherein the housing further comprises one or more channels for receiving at least one of: a heating element and a cooling element.

12. The non-invasive device according to claim 1, further comprising a mounting mechanism for mounting the non-invasive device to the face of the subject.

13. The non-invasive device according to claim 1, further comprising a channel to receive tubing for providing fluids.

14. The non-invasive device according to claim 1, further comprising connecting elements for receiving one or more add-on devices.

15. A non-invasive device for measuring one or more respiratory flow parameters, comprising:
a first sensor for measuring one or more respiratory flow parameters; and
a housing anatomically shaped to be attached to a face of a subject externally to the respiratory orifices,
wherein the housing comprises:
one or more flow directing elements for directing at least a portion of the respiratory flow to at least a first predetermined location, wherein the first predetermined location is configured to hold at least the first sensor and wherein each of the one or more flow directing elements includes at least one open conduit.

16. The non-invasive device of claim 15, comprising a second sensor, wherein the one or more flow directing elements is for directing at least a portion of the respiratory flow to at least a second predetermined location, wherein the second predetermined location is configured to hold the second sensor.

17. The non-invasive device of claim 16, wherein the first sensor is for measuring a first type of respiratory flow parameters and the second sensor is for measuring a second type of respiratory flow parameters.

18. The non-invasive device of claim 16, wherein the first sensor is for measuring a first type of respiratory flow parameter at the first predetermined location and the second sensor is for measuring the first type of respiratory flow parameter at a second predetermined location.

19. A method of measuring one or more respiratory flow parameters, comprising:
mounting a non-invasive device to a face of a subject, the non-invasive device comprising at least one sensor for measuring one or more respiratory flow parameters, and a housing anatomically shaped to be attached to a face of a subject externally to the respiratory orifices, wherein the method comprises:
directing at least a portion of the respiratory flow to at least a first predetermined location using one or more flow directing elements that includes at least one open conduit, wherein the first predetermined location is configured to hold the at least one sensor; and
receiving measurements from the at least one sensor.

20. The method of claim 19, further comprising:
controlling at least one of a heating and a cooling element included in the non-invasive device to control a temperature of at least one of: the housing and the at least one sensor.
